(19) United States
(12) Patent Application Publication
(10) Pub. No.: US 2026/0231916 A1
(43) Pub. Date: Aug. 13, 2026

(54) SYSTEM AND METHOD FOR ORAL VACCINATION OF ANIMAL POPULATIONS VIA TRANSGENIC INSECTS

(71) Applicant: Future Fields Cellular Agriculture and Research LTD., Edmonton (CA)

(72) Inventor: Matthew Anderson-Baron, Edmonton (CA)

(21) Appl. No.: 19/535,460

(22) Filed: Feb. 10, 2026

Related U.S. Application Data
(60) Provisional application No. 63/756,400, filed on Feb. 10, 2025.

(51) Int. Cl. A01K 67/68; A01K 67/36; A61K 39/00; C12N 15/85

(52) U.S. Cl. CPC A01K 67/68; A01K 67/36; A61K 39/00; C12N 15/8509; A01K 2217/052; A01K 2227/706; A01K 2267/01; A61K 2039/542; A61K 2039/552; C12N 2015/8518

(57) ABSTRACT

One variation of a method includes: identifying a target pathogen associated with disease in a target aquatic species; accessing a vaccine antigen sequence encoding a target antigen for the target pathogen, the target antigen configured to trigger a target immune response in the target aquatic species responsive to exposure to the target pathogen; generating a transgenic Drosophila strain expressing the target antigen by inserting the vaccine antigen sequence into a genome of a base Drosophila species; cultivating a population of the transgenic Drosophila strain to produce an amount of Drosophila biomass comprising the target antigen; processing the amount of Drosophila biomass into a vaccine feed configured for oral administration to a population of the target aquatic species; and administering the vaccine feed to the population of the target aquatic species via oral delivery to vaccinate the population of the target aquatic species against the target pathogen.

### CROSS-REFERENCE

This Application claims the benefit of U.S. Provisional Application No. 63/756,400, filed on 10 Feb. 2025, which is incorporated in its entirety by this reference.

This Application is also related to U.S. patent application Ser. No. 19/209,586, filed on 15 May 2025, which: claims the benefit of U.S. Provisional Application No. 63/648,114, filed on 15 May 2024, which is incorporated in its entirety by this reference; and is a continuation-in-part of U.S. patent application Ser. No. 18/075,362, filed on 5 Dec. 2022, which is a continuation-in-part of U.S. patent application Ser. No. 17/086,226, filed on 30 Oct. 2020, which claims the benefit of U.S. Provisional Application No. 62/927,788, filed on 30 Oct. 2019, each of which is incorporated in its entirety by this reference.

### TECHNICAL FIELD

This invention relates generally to the field of animal disease management and, more specifically, to a new and useful method for oral vaccine delivery via transgenic insects in the field of animal disease management.

### BRIEF DESCRIPTION OF THE FIGURES

FIG. 1 is a flowchart representation of a method;

FIG. 2 is a flowchart representation of one variation of the method; and

FIG. 3 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

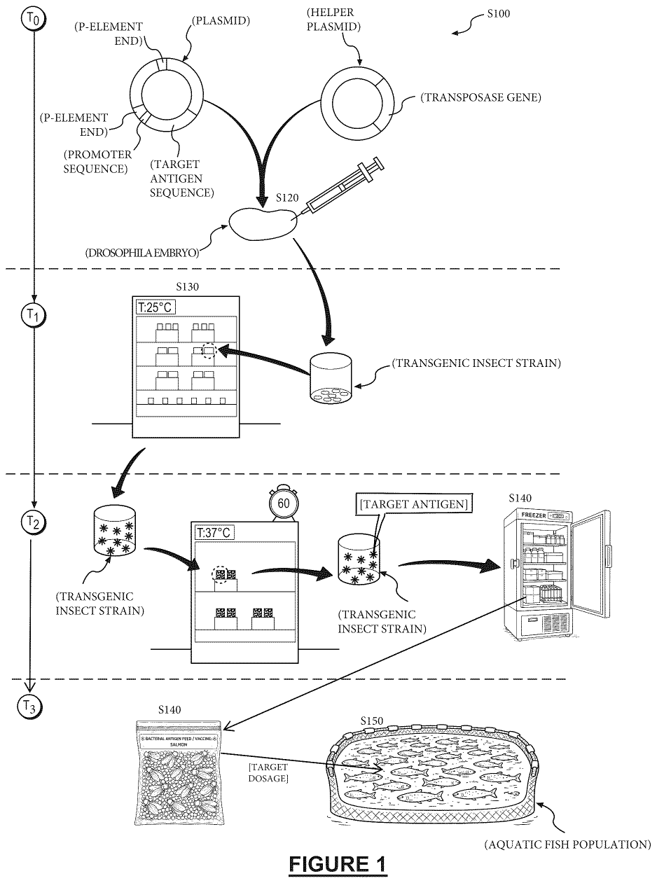

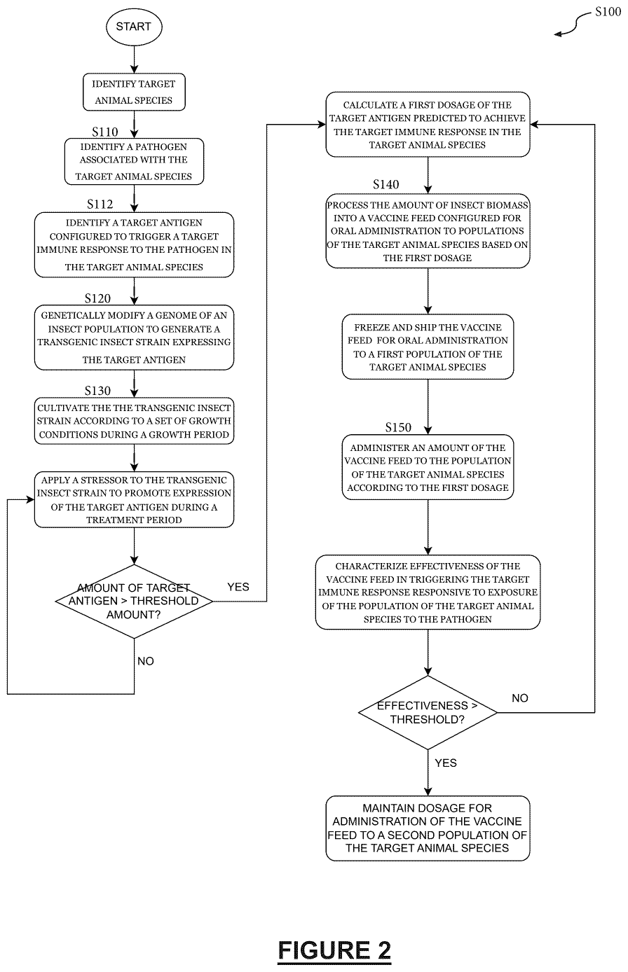

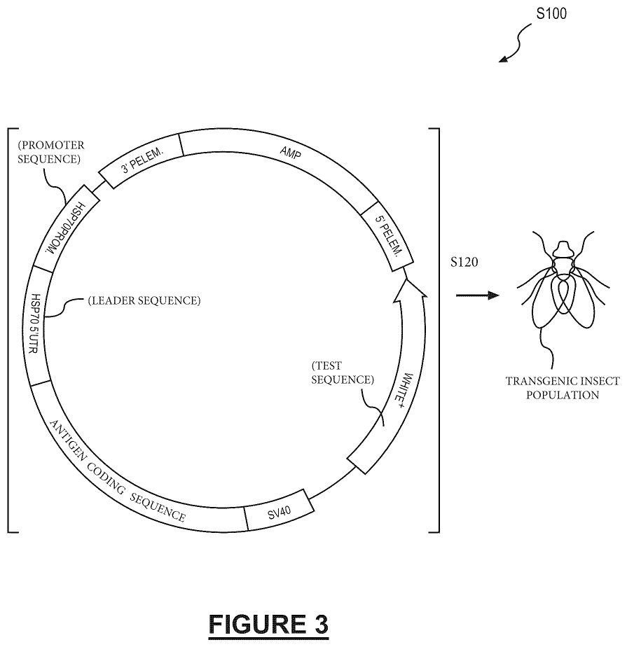

As shown in FIGS. 1-3, a method S100 includes: identifying a target pathogen associated with disease in a target animal species in Block S110; accessing a vaccine antigen sequence encoding a target antigen—configured to trigger a target immune response in the target animal species responsive to exposure to the target pathogen—for the target pathogen in Block S112; generating a transgenic insect strain expressing the target antigen by inserting the vaccine antigen sequence into a genome of a base insect species in Block S120; cultivating a population of the transgenic insect strain to produce an amount of insect biomass including the target antigen in Block S130; processing the amount of insect biomass into a vaccine feed configured for oral administration to a population of the target animal species in Block S140; and, at a first time, administering the vaccine feed to the population of the target animal species via oral delivery to vaccinate the population of the target animal species against the target pathogen in Block S150.

In one variation, the method further includes, at a second time succeeding the first time by a target duration, characterizing an immune response—representing magnitude of protection against the target pathogen—of the population of the target animal species responsive to exposure to the target pathogen in Block S160.

### 1.1 Method: Drosophila+Aquaculture

As shown in FIGS. 1-3, one variation of the method S100 includes: identifying a target pathogen associated with disease in a target aquatic species in Block S110; accessing a vaccine antigen sequence encoding a target antigen—configured to trigger a target immune response in the target aquatic species responsive to exposure to the target pathogen—for the target pathogen in Block S112; generating a transgenic Drosophila strain expressing the target antigen by inserting the vaccine antigen sequence into a genome of a base Drosophila species in Block S120; cultivating a population of the transgenic Drosophila strain to produce an amount of Drosophila biomass including the target antigen in Block S130; processing the amount of Drosophila biomass into a vaccine feed configured for oral administration to a population of the target aquatic species in Block S140; at a first time, administering the vaccine feed to the population of the target aquatic species via oral delivery to vaccinate the population of the target aquatic species against the target pathogen in Block S150; and, at a second time succeeding the first time by a target duration, characterizing an immune response in the population of the target aquatic species indicating protection against the target pathogen in Block S160.

### 2. Applications

Generally, as shown in FIG. 1, Blocks of the method S100 can be executed by a system—such as a local or remote computer system in conjunction with a user (e.g., a laboratory technician, an operator)—to: cultivate a transgenic strain of insects configured to express a target antigen corresponding to a particular disease (e.g., bacterial, viral, fungal, and/or parasitic disease) in an animal species; control generation of this target antigen within the transgenic strain of insects; and formulate and process an animal feed—including insects from the transgenic strain of insects—for oral administration to animals in the animal species for protection against the particular disease. Therefore, when an animal in the animal species consumes the animal feed containing insects from the transgenic strain of insects, presence of the target antigen in the consumed animal feed triggers generation of antibodies—that target the particular disease—by the animal's immune system.

In particular, Blocks of the method S100 can be executed to: insert antigen genes—targeting specific pathogens and/or disease—into insect genomes and thus generate stable transgenic insect lines that express vaccine antigens across successive generations; cultivate transgenic insects under particular environmental conditions in order to promote generation of antigens; harvest insect biomass containing one or more target antigens at threshold expression levels; freeze insect biomass to preserve antigen functionality and enable global distribution; formulate frozen insect biomass into feed compositions compatible with feeding practices of a target animal species (e.g., aquaculture, poultry, livestock); dose animal populations by mixing transgenic insect biomass with standard feed and thus deliver vaccine antigens directly to animal populations via natural feeding behaviors; and, therefore, achieve protective immunity within these animal populations with reduced stress, labor costs, and mortality associated with injection-based vaccination.

In particular, in one implementation, a genome of a transgenic strain of insects (e.g., a population of Drosophila) can be genetically-modified to include: a target sequence encoding for the target antigen; and a set of regulatory sequences (e.g., a promoter sequence) upstream the target sequence and configured to regulate expression of the target sequence and, therefore, regulate production of the target antigen in insects in the transgenic strain of insects. Additionally or alternatively, the genome of the transgenic strain of insects can be genetically modified to include a particular promoter sequence—associated with a particular stressor (e.g., heat shock)—upstream the target sequence and configured to drive expression of the target sequence responsive to insect exposure to the particular stressor. For example, the transgenic strain of insects can be genetically modified to incorporate a heat-shock promoter configured to drive antigen expression only when insects are exposed to elevated temperatures (e.g., above a threshold temperature), thereby enabling increased regulation of antigen expression in the transgenic strain of insects.

The transgenic strain of insects can then be cultivated and subjected to various conditions—such as including application of a stressor—in order to yield a target amount of the target antigen expressed by insects in the transgenic strain of insects. The transgenic strain of insects—including this target amount of the target antigen—can then be harvested for preparation of an animal feed configured for consumption by animals in the target animal species. For example, the transgenic strain of insects can be processed by: immediately freezing insects to preserve antigen structure and create a frozen biomass of insects; lyophilizing insects to create a shelf-stable powdered biomass with extended storage life; grinding frozen or lyophilized insects into a particulate form compatible with mixing into animal feed; and/or maintaining insects whole for direct feeding to animals of an animal species that consumes whole insects.

In one implementation, Blocks of the method S100 can be executed to develop an oral vaccination—including genetically-modified insects configured to express a target antigen—for consumption by aquatic fish populations (e.g., farmed fish) that may be more susceptible to infectious disease due to environmental conditions inhabited by these populations and characterized by relatively high stocking densities, diminished oxygen levels, sub-optimal water quality, and/or elevated pathogen loads. Therefore, in order to limit spread of infectious disease in these populations—and significantly reduce population loss and associated costs—the transgenic strain of insects can be genetically modified to express specific antigens—targeting diseases experienced by aquatic fish populations in varying environmental conditions—and can be processed to develop a fish feed incorporating insects from the transgenic strain of insects and, therefore, incorporating these target antigens.

Therefore, Blocks of the method S100 can be executed to: orally vaccinate aquatic fish populations via consumption of insect biomass that fish naturally consume as food; eliminate a requirement for stress-inducing injection or immersion vaccination methods; enable rapid deployment of vaccines in response to disease outbreaks; and dramatically reduce mortality rates in aquatic fish populations due to pathogen exposure.

Generally, Blocks of the method S100 are described below as executed to generate an oral vaccine for consumption by an aquatic fish population. However, Blocks of the method S100 can be executed to generate an oral vaccine for consumption by populations of various animal species, such as including aquatic fish populations, poultry populations, livestock populations, etc.

### 2.1 Applications: “Bio-Encapsulation” & Antigen Protection

The system leverages natural bio-encapsulation of antigens within insect tissue to protect antigens during transit through the fish digestive system. In particular, antigens expressed within insect cells benefit from intrinsic protection by cellular membranes, cytoskeletal structures, and extracellular cuticle. The vaccine antigen is biologically encapsulated in the insect larvae, which protects the antigen from degradation during digestion, thereby enabling a progressive release of antigens from insect biomass as it transits the digestive tract.

This progressive release of antigens from insect biomass as it transits the digestive tract—combined with protection from complete enzymatic degradation—enables increased quantities of the target antigen to reach regions of the fish digestive system where absorption occurs and immune responses are initiated.

### 2.2 Drosophila

In one implementation, the transgenic strain of insects is a population of Drosophila genetically modified to produce a target antigen.

“Drosophila” is referred to herein as a genus of flies belonging to the family Drosophilidae, members of which may be referred to as “small fruit flies,” “pomace flies,” “vinegar flies,” and/or “wine flies.” A population of Drosophila can include flies of a particular species, such as D. melanogaster (or “the common fruit fly”), D. immigrans, D. innubila, D. funebris, D. neotestacea, D. virilise, D. hydei, etc. For example, a population of D. melanogaster can be cultivated and configured for production of recombinant proteins. For example, the transgenic strain of insects can include a population of Drosophila melanogaster.

A genome of the population of Drosophila can be genetically modified to include a target sequence encoding for the target antigen. Once the genome is genetically modified and inserted into the population of Drosophila (e.g., as embryos), the population of Drosophila can grow, reproduce, and propagate the genome through each subsequent generation of the population. Further, during a growth period, the population of Drosophila can be fed a simple and inexpensive diet including cornmeal-based gelatinous foods. Therefore, the population of Drosophila including the genetically-modified genome can be relatively inexpensive to maintain. In addition, because Drosophila exhibit relatively short lifespans (e.g., less than 50 days), new generations can be rapidly produced, enabling frequent collection of the target antigen from the population of Drosophila. Therefore, a large quantity of the target antigen can be collected over a relatively short period of time.

Unlike cell-based systems, Drosophila include immune systems and therefore exhibit lower risk of contamination within the population of Drosophila. Further, unlike bacterial systems, Drosophila can implement post-translational modification to produce biologically active recombinant proteins (i.e., biologically active target antigens).

In one implementation, the genome of the population of Drosophila can be genetically modified via P-elements, present in Drosophila, which are transposable elements that enable genes to move within the genome. These P-elements can cooperate with transposons to insert an exogenous gene inserted within a vector into the genome of the Drosophila population. These P-elements of Drosophila therefore enable insertion of an exogenous gene (i.e., the target sequence) and expression of a resulting protein (i.e., the target antigen) encoded by the exogenous gene into the genome of the population of Drosophila. Alternatively, in another implementation, the genome of the population of Drosophila can be genetically modified via a site-specific integrase system.

### 3. Vaccine Development

Generally, a transgenic strain of insects can be genetically modified to express a target antigen corresponding to a target disease (e.g., bacterial, viral, fungal, or parasitic disease) associated with aquatic fish populations.

For example, the transgenic strain of insects can be modified to express a target antigen configured to trigger an immune response—responsive to exposure to a bacterial pathogen by an aquatic species (e.g., tilapia, trout, or catfish) when vaccinated via consumption of insects in the transgenic strain of insects.

In another example, the transgenic strain of insects can be modified to express a set of target antigens—each configured to trigger an immune response (e.g., a as a “multivalent” vaccine)—responsive to exposure to a host of pathogens by an aquatic species (e.g., salmon, sea bass) when vaccinated via consumption of insects in the transgenic strain of insects.

### 3.1 Genetic Modification of Insect Population

A transgenic strain of insects can be genetically modified to produce a particular target antigen. In particular, insect cells can be genetically modified to include a target sequence encoding for the target antigen, such that when the target sequence is expressed, the insect cells generate the target antigen.

In one implementation, the transgenic strain of insects can define a genetically-modified line of insects including multiple generations. In this implementation, a first generation of insects can be: genetically modified to produce a target antigen and then propagated to produce additional generations of insects configured to produce the target antigen.

For example, a first generation can be genetically modified to produce a target antigen. In particular, a target sequence encoding for the target antigen can be inserted into a genome of the first generation of insects. Once this target sequence is stably integrated into the genome, this first generation can be propagated in cages over a propagation period, during which adult female insects in the first generation may lay eggs. These eggs develop into embryos, which eventually hatch, thereby introducing new genetically-modified insect larvae defining a second generation of insects. The genome of this second generation thereby includes the target sequence—inherited from the first generation—encoding for the target antigen. The target antigen can then be produced and collected from the second generation via the methods and techniques described below. Further, once the insect larvae in the second generation mature into adult insects, adult female insects in the second generation lay eggs and continue propagation of this line of genetically-modified insects. Therefore, in this implementation, once the genome of the first generation of insects is genetically modified to include the target sequence, this genome can be propagated through the line of insects, over multiple generations, without further genetic modification.

### 3.1.1 Regulatory Sequences

The genome of the transgenic strain of insects can be genetically modified to include a set of regulatory sequences upstream the target sequence. These regulatory sequences can be leveraged to control expression of the target sequence and therefore control production of the target antigen expressed by the target sequence.

### 3.1.2 Promoter Sequence

In one implementation, the set of regulatory sequences can include a promoter sequence coupled to the target sequence. The promoter sequence can be associated with a particular stressor (e.g., heat-shock, cold-shock, nutrient-deprivation, dehydration, UV exposure), such that application of the particular stressor to the transgenic strain of insects activates expression of the promoter sequence. For example, the genome of the transgenic strain of insects can be genetically modified to include an HSP70 promoter sequence upstream the target sequence, such that activation of the HSP70 promoter leads to expression of the target sequence and thereby generation of the target antigen. The HSP70 promoter sequence can activate in the presence of a heat-shock stressor in order to express heat-shock proteins which may protect cells from damage caused by the heat-shock stressor. More specifically, in response to a particular dosage of a heat-shock stressor, the HSP70 promoter sequence—bound by a set of transcription factors—can initiate transcription of sequences immediately downstream the HSP70 promoter sequence. Therefore, by coupling the target sequence to the HSP70 promoter sequence immediately upstream the target sequence, a heat-shock stressor can be implemented to control transcription of the target sequence.

Therefore, in the preceding example, a heat-shock stressor can be applied to the transgenic strain of insects to increase production of the target sequence. Thus, in this implementation, the genome of the transgenic strain of insects can be genetically modified to include: a target sequence encoding for a particular target antigen; and a promoter sequence upstream the target sequence and associated with a particular stressor. By coupling the target sequence with a promoter sequence upstream of the target sequence, expression of the promoter sequence can be leveraged to control expression of the target sequence and, therefore, to control generation of the target antigen.

In another example, the transgenic strain of insects can be genetically modified to include a set of regulatory sequences including a promoter sequence linked to a cold-shock stressor. In particular, the transgenic strain of insects 101 can include: a promoter sequence (e.g., coding for a heat-shock protein) associated with a stress response (e.g., cold stress response) of the transgenic strain of insects (e.g., Drosophila) to the cold-shock stressor, such that the promoter sequence exhibits increased expression responsive to application of the cold-shock stressor; and a target sequence linked (i.e., downstream) to the promoter sequence, such that expression of the promoter sequence promotes expression of the target sequence. Therefore, in this example, the cold-shock stressor can be applied to the transgenic strain of insects to trigger increased expression of the promoter sequence (e.g., coding for a particular heat-shock protein associated with a cold-stress response) and thereby trigger increased production of the target sequence.

In yet another example, the transgenic strain of insects can be genetically modified to include a set of regulatory sequences including a promoter sequence linked to a UV stressor (i.e., an Ultraviolet radiation stressor or “UVR” stressor). In particular, in this example, the transgenic strain of insects 101 can include: a promoter sequence (e.g., coding for a particular heat-shock protein) associated with a stress response (e.g., a UV-stress response) of the transgenic strain of insects to the UV stressor, such that the promoter sequence exhibits increased expression responsive to application of the UV stressor; and a target sequence linked (i.e., downstream) to the promoter sequence, such that expression of the promoter sequence promotes expression of the target sequence. Therefore, in this example, the UV stressor can be applied to the transgenic strain of insects to trigger increased expression of the promoter sequence (e.g., coding for a particular heat-shock protein associated with a UV-stress response) and thereby trigger increased production of the target sequence. In yet another example, the transgenic strain of insects can be similarly genetically modified to include a set of regulatory sequences including a promoter sequence (e.g., encoding for a particular heat-shock protein associated with a nutrient-stress response) linked to a nutrient stressor (e.g., a nutrient deficiency or other dietary stressor).

However, the transgenic strain of insects can be genetically modified to include promoter sequence (and/or additional regulatory sequences)—linked to downstream target sequence—associated with other stressor types, such as environmental conditions, chemical agents, and/or biological agents.

### 3.1.3 Inducible Expression

In one variation, the system can engineer a transgenic strain of insects with inducible expression systems enabling controlled antigen production in response to an external stimuli.

For example, in this variation, the system can: incorporate a heat-shock promoter configured to drive antigen expression only when insects are exposed to elevated temperatures (e.g., above a threshold temperature); rear insects under moderate and/or room temperatures during growth and development, during which expression of the antigen gene remains at lower expression levels and/or inactive; at a defined developmental stage, expose insects to heat shock to induce robust antigen expression; and harvest insects at a time associated with peak antigen concentration in insects in the transgenic strain following exposure to heat shock. Therefore, the system can enable temporally-controlled antigen production configured to maximize antigen production in insects in the transgenic strain of insects.

### 3.1.4 Example

In one example, a population of Drosophila can be genetically modified to produce a target antigen. In this example, the target sequence (i.e., coding sequence)—coding for the target antigen (e.g., a target recombinant protein)—is cloned via restriction enzyme cloning into a pUAST plasmid including a multiple cloning site (or “MCS”). The target sequence is then amplified by PCR with the addition of restriction cut sites for one or two of the restriction enzymes added onto 5′ and 3′ amplification primers, thereby enabling insertion of the target sequence 112 into the pUAST plasmid vector at the MCS via a T4 DNA ligase.

The resulting plasmid vector—including the target sequence (e.g., the recombinant protein sequence)—can then be transformed into chemically competent bacterial cells for propagation. In this example, this transformation can be performed under ampicillin selection due to the presence of an ampicillin resistance gene in the pUAST vector. After the plasmid vector is thus propagated in the bacterial culture, the plasmid DNA is extracted and purified. Insertion of the target sequence (e.g., the recombinant protein-coding sequence) into the pUAST vector can then be verified by sequencing upstream and downstream from the insertion site.

Upon confirmation of insertion into the plasmid (i.e., the pUAST plasmid vector), the target sequence can be stably inserted into the Drosophila genome. For example, the target sequence can be stably inserted into the Drosophila genome via a P-element transposon present in the plasmid vector. In particular, the pUAST vector includes P-element sites that—in the presence of a transposase—enable stable integration of exogenous DNA into the Drosophila genome. The target sequence can be inserted via germline transformation through microinjection of the modified plasmid DNA (i.e., the modified pUAST plasmid vector) and a helper plasmid—including transposase—into a recipient Drosophila embryo. In this example, the plasmid DNA (e.g., the modified pUAST plasmid vector) is delivered to the posterior pole of the syncytial blastoderm, at which precursors of the germ cells are formed. Thus, upon cellularization of the embryo, the plasmid DNA (e.g., the modified pUAST plasmid vector)—including P-elements—is integrated into the genome of the germ cells via activity of the transposase.

### 4. Insect Cultivation & Harvesting

Generally, the transgenic strain of insects can be: cultivated during a growth period—according to a set of growth conditions—to promote growth of insects in the transgenic strain of insects and expression of the target antigen; and harvested—following expression of the target antigen at expression levels exceeding a threshold level—for generation of an animal feed configured to be fed to a target animal population as an oral vaccination.

### 4.1 Insect Cultivation

Generally, during a growth period, the transgenic strain of insects can be cultivated according to a set of growth conditions. During the growth period, the transgenic strain of insects can be grown from a first stage (e.g., embryo) to a second stage (e.g., larvae, pupae, adult) over a duration of the growth period. During this growth period, the transgenic strain of insects can be subjected to a set of growth conditions (e.g., temperature, nutrient availability) configured to increase survival rates of insects in the transgenic strain of insects for the duration of the growth period.

In one implementation, the transgenic strain of insects can be cultivated during the growth period spanning a transition of the transgenic strain of insects from an embryonic stage to a larval stage. For example, during the growth period, genetically-modified embryos of a population of Drosophila can be incubated at a set temperature of approximately (e.g., within two degrees Celsius) twenty-five degrees Celsius. Once these embryos hatch to produce larvae, the population of Drosophila can be fed a particular diet (e.g., of cornmeal-based gelatinous foods). The population of Drosophila can be subjected to these conditions throughout the duration of the growth period, such as without introduction of a stressor.

In one implementation, the growth period can define a duration corresponding to transition to a particular life stage. For example, the transgenic strain of insects in the preceding example can be incubated at the set temperature over a first duration (e.g., 4 days) spanning a transition from embryo to a third instar larval stage. In this example, by terminating the growth period at the third instar larval stage, an amount (e.g., concentration, quantity) of the target antigen generated by the transgenic strain of insects can be quickly harvested before degradation of the target antigen. Alternatively, in another example, the transgenic strain of insects can be incubated at the set temperature over a second duration (e.g., 5 days) spanning a transition from embryo, to larvae, to pupae. Alternatively, in yet another example, the transgenic strain of insects can be incubated at the set temperature over a third duration (e.g., 10 days, 15 days, 30 days) spanning a transition from embryo, to larvae, to pupae, to adult. In this example, the third duration can be selected to enable egg laying by female insects (e.g., female Drosophila) prior to termination of the growth period for continued propagation of the target sequence—encoding for the target antigen—within the transgenic strain of insects.

### 4.2 Insect Harvesting

Generally, the transgenic strain of insects can be harvested following the growth period and/or one or more treatment cycles, including application of the stressor (e.g., heat shock treatment).

In one implementation, the transgenic strain of insects can be harvested once a particular amount (e.g., exceeding a defined threshold amount) of the target antigen has been produced. In particular, in this implementation, Blocks of the method S100 can include: predicting a first amount (e.g., concentration, quantity, proportion) of the first target antigen produced within the transgenic strain of insects during the growth and/or treatment periods; and, in response to the first amount exceeding a threshold amount defined for the target antigen, harvesting the transgenic strain of insects. For example, the transgenic strain of insects can be harvested in response to an amount (e.g., quantity, proportion, concentration) of the target antigen produced within the transgenic strain of insects exceeding a threshold amount, such as a threshold concentration of one percent, two percent, five percent, fifteen percent, thirty percent, etc.

Additionally or alternatively, in the preceding implementation, in response to the amount of the first target antigen falling below the threshold amount, a subsequent stressor cycle—including application of a stressor configured to drive expression of the first target antigen by the transgenic strain of insects—can be implemented in order to trigger further production of the first target antigen in the transgenic strain of insects.

### 5. Biomass Collection & Feed Formulation

Generally, in response to harvesting the transgenic strain of insects, these insects can be formulated into an animal feed configured for consumption by the target animal population as an oral vaccination.

### 5.1 Quality Control

In one variation, the system can implement quality control protocols configured to regulate and/or control antigen expression across production batches of the transgenic population of insects.

In particular, in response to harvesting of the transgenic strain of insects, a sample insect population can be extracted from the transgenic strain of insects for quality control analysis. The system—such as including a human user interfacing with the computer system—can: sample each production batch of the transgenic population of insects at harvest; extract proteins from sampled insects in each production batch; quantify antigen concentration in these sampled insects (e.g., via standardized immunoassays); verify antigen structural integrity (e.g., via SDS-PAGE or Western blot analysis); test for absence of contaminating pathogens that may affect animal health; measure moisture content, protein content, and/or lipid content of insect biomass; and compare each of these batch specification to a set of pre-defined target specifications. The system can then verify production batches that meet the set of pre-defined target specifications, such as including: an antigen concentration within a threshold difference (e.g., between 80-120%) of a target antigen concentration; absence of detectable contamination; and/or a moisture content compatible with processing and storage. Furthermore, the system can trigger: further processing of verified batches of the transgenic strain of insects; and trigger quarantining and/or discarding of unverified batches—not meeting the target set of specifications—for further investigation, reprocessing, and/or disposal.

### 5.2 Vaccine Feed Formulation & Processing

Generally, the transgenic strain of insects can be harvested during a harvest period succeeding the growth period.

In particular, in one implementation, the transgenic strain of insects can be: collected and washed to remove substrate residues and external contaminants; and selectively processed in preparation for packaging and/or incorporation in an animal feed. For example, the transgenic strain of insects can be harvested and processed by: immediately freezing insects to preserve antigen structure and create frozen biomass, lyophilizing insects to create shelf-stable powdered biomass with extended storage life, grinding frozen or lyophilized insects into particulate form compatible with mixing into fish feed; and/or maintaining insects whole—for direct feeding to fish species that consume whole insects—and freezing these whole insects.

The harvested and processed transgenic insect population—such as including whole, frozen, transgenic insects—can then be packaged according to a set of packaging conditions configured to prevent moisture absorption and oxidation. The packaged transgenic insect populations can then: be labelled with an identity of the target antigen, a concentration of the target antigen, a production date, a batch number, recommended storage conditions, expiration date, etc.; be stored according to a set of target storage condition configured to maintain antigen functionality during distribution to aquaculture facilities.

The system can thus harvest the transgenic strain of insects, freeze the insect biomass, and ship frozen larvae to aquaculture facilities.

### 5.2.1 Dosing

In one implementation, the system can calculate a mass of transgenic insect biomass required to deliver a target antigen dose to the animals in the target animal population.

For example, for an aquatic fish population, the system can calculate a mass of transgenic insect biomass required per fish based on: an antigen concentration of the target antigen in an amount of transgenic insect biomass; a target antigen dose per fish; and an expected feed consumption per fish. The system can also select and/or access a feed incorporation strategy for administering biomass of transgenic insects to the aquatic fish population. For example, the system can select and/or access a feed incorporation strategy such as including: whole insect feeding for fry and fingerlings that readily consume small whole insects; biomass powder mixed with pelleted feed for larger fish or production systems that implement automated feeding; encapsulated biomass—protected by additional coating—for species with aggressive digestive systems; etc.

The system can then: mix calculated quantities of transgenic insect biomass with base feed formulation (e.g., if required) according to the selected feed incorporation strategy to generate a transgenic insect feed; and ensure homogeneous distribution of transgenic insect biomass throughout the transgenic insect feed. Furthermore, the system can: pelletize or extrude combined formulation if producing pelleted feed; dry process feed to achieve a target moisture content configured to prevent microbial growth.

### 6. Vaccine Deployment

Generally, the system—such as in combination with a farmer and/or feed administrator—can administer the transgenic insect biomass (or “insect vaccine”) to the target animal population via direct feeding of transgenic insects to animals, such as according to a particular schedule and dosage.

### 6.1 Vaccination Timing

In one implementation, the system can suggest and/or implement a vaccination schedule based on animal developmental stage and immune system maturation.

The system can: identify an earliest developmental stage at which animals possess functional adaptive immune systems capable of generating protective antibody responses; schedule primary vaccination after immune system maturation and before significant disease exposure risk; suggest and/or implement vaccination during early production phases when animal populations are concentrated and feeding is closely controlled; for diseases with high infection pressure, administer booster vaccinations several weeks after primary vaccination to enhance antibody titers and immunological memory; schedule final vaccination to occur at least several weeks before anticipated disease exposures to allow adequate immune response development; and coordinate vaccination timing with other species management activities in order to minimize cumulative stress.

### 6.2 Dosage

In one implementation, the system can calculate a vaccine dosage configured to achieve a target immune response in the target animal population.

In particular, in this implementation, the system can: count or estimate a quantity of animals in the target animal population; access an average animal weight defined for animals in the target animal population; access a feeding rate defined for the target animal population; access a vaccination period duration defined for the target antigen, such as a single dose fed to the animal population in one feeding and/or multiple doses spread across several feedings; calculate a total feed requirement based on the quantity of animals, the average animal weight, the feeding rate, and the vaccination period duration; calculate vaccine feed quantity delivering target antigen dose to each animal over the vaccination period; etc.

### 6.3 “Vaccine” Administration

In one implementation, the system—such as in combination with a farmer and/or feed administrator—can administer vaccine feed to animal populations via protocols designed for antigen delivery and immune response induction.

The system—such as in coordination with a farmer and/or feed administrator—can: mix transgenic insect biomass with standard feed at calculated ratios; distribute vaccine feed to animals during normal feeding times; monitor animal behavior and feed consumption to ensure adequate vaccine intake; track vaccination progress across the animal population; record vaccination dates and doses for regulatory compliance; and collect samples from vaccinated animals to verify immune response development.

Therefore, the transgenic insect biomass can be readily fed to the target animal population via standard feeding procedures already implemented for the target animal population.

### 7. Closing the Loop

In one variation, the method S100 further includes characterizing effectiveness of the vaccine following administration of the insect vaccine to the target animal population.

In particular, following administration of the vaccine feed composition at the first time, the system can characterize an immune response of the target animal population indicative of protection against the target pathogen.

In one implementation, the system can characterize vaccine effectiveness by measuring a set of biological indicators associated with immune activation in animals of the target animal population. For example, the system can access and/or measure: presence or concentration of antigen-specific antibodies; activation of innate immune responses; activation of adaptive immune pathways; expression of immune-related genes; and/or other biomarkers associated with immunological protection against the target pathogen.

Additionally or alternatively, the system can characterize vaccine effectiveness through exposure of the target animal population to the target pathogen following administration of the insect vaccine to the target animal population. In this implementation, the system can monitor vaccination outcomes including: survival rates; disease incidence; disease severity; pathogen load; and/or recovery time relative to an unvaccinated population and/or control population. Therefore, the system can characterize effectiveness of the vaccine based on differential outcomes observed between vaccinated and control populations.

In one variation, the system can characterize vaccine effectiveness across multiple timepoints following administration of the vaccine feed composition. For example, the system can evaluate immune response persistence and/or duration of protection by characterizing immune response metrics at multiple timepoints succeeding administration of the insect vaccine. Therefore, the system can assess durability of protection conferred by the vaccine delivered via the transgenic insect biomass.

Therefore, Blocks of the method S100 can be executed to verify that administration of the insect vaccine induces a protective immune response in the target animal population and reduces susceptibility to disease caused by the target pathogen.

### 8. Variation: Poultry

In one implementation, the system can develop transgenic insect vaccines for a poultry species that naturally consumes insects.

In particular, in this implementation, the system can: identify high-priority poultry diseases, such as including avian influenza, Newcastle disease, infectious bronchitis, and infectious bursal disease; engineer transgenic insect strains expressing antigens that target these common poultry pathogens; implement the methods and techniques described above to formulate transgenic insect biomass into poultry feed compositions; administer vaccine feed to chickens, turkeys, ducks, and other poultry during rearing; evaluate efficacy through challenge studies measuring protection against poultry diseases; and thus achieve stress-free oral delivery and rapid deployment of vaccines targeting high-priority poultry diseases, thereby minimizing costs due to poultry loss and/or implementation of higher stress vaccination techniques. Therefore, the system can: enable vaccination of large flocks where individual injection is impractical; support vaccination of free-range and backyard flocks with limited infrastructure; provide rapid response to emerging disease threats; and reduce reliance on antibiotics in poultry production.

### 9. Variation: Livestock Species

In one implementation, the system can develop transgenic insect vaccines for swine and other livestock species.

In particular, in this implementation, the system can: identify target diseases in swine and/or livestock, such as including porcine reproductive and respiratory syndrome, swine influenza, and porcine circovirus; and implement the methods and techniques described above to formulate transgenic insect biomass into swine feed compositions; administer vaccine feed to swine, cattle, sheep, goats; etc. Therefore, the system can: enable stress-free vaccination of large swine herds; reduce handling labor and associated costs; improve animal welfare by eliminating injection-related stress; and provide a platform for rapid response to emerging swine diseases.

As a person skilled in the art will recognize from the previous detailed description and from the figures and claims, modifications and changes can be made to the embodiments of the invention without departing from the scope of this invention as defined in the following claims.

I claim:

1. A method comprising:
identifying a target pathogen associated with disease in a target aquatic species;
accessing a vaccine antigen sequence encoding a target antigen for the target pathogen, the target antigen configured to trigger a target immune response in the target aquatic species responsive to exposure to the target pathogen;
generating a transgenic Drosophila strain expressing the target antigen by inserting the vaccine antigen sequence into a genome of a base Drosophila species;
cultivating a population of the transgenic Drosophila strain to produce an amount of Drosophila biomass comprising the target antigen;
processing the amount of Drosophila biomass into a vaccine feed configured for oral administration to a population of the target aquatic species; and
at a first time, administering the vaccine feed to the population of the target aquatic species via oral delivery to vaccinate the population of the target aquatic species against the target pathogen.

2. The method of claim 1, further comprising, at a second time succeeding the first time by a target duration, characterizing an immune response in the population of the target aquatic species responsive to exposure to the target pathogen, the immune response indicating a magnitude of protection against the target pathogen.
